(19) United States
(12) Patent Application Publication
(10) Pub. No.: US 2004/0028555 A1
(43) Pub. Date: Feb. 12, 2004

(54) Sterile connection

(75) Inventor: Stephen Warburton-Pitt, Andover, NJ (US)

Correspondence Address: David L. Davis, 203 Main Street, Metuchen, NJ, 08840, US

(73) Assignee: Truseal USA, Inc.

(21) Appl. No.: 10/216,021

(22) Filed: Aug. 9, 2002

(51) Int. Cl. A61L 2/00; B01J 19/00

(52) U.S. Cl. 422/26; 422/1; 422/40; 422/292

(57) ABSTRACT

An assembly and a method for providing a sterile connection between a vessel and a bag. The assembly includes a previously sterilized bag connected to tubing which is clamped closed. The tubing is connected to the vessel. The vessel and the tubing distally of the clamp are sterilized together. The clamp is then opened to allow material to pass from the vessel to the bag in a sterile environment.

### BACKGROUND OF THE INVENTION

This invention relates to the provision of a sterile connection between vessels which may be used in pharmaceutical manufacturing.

During the manufacture of a pharmaceutical product, it is often required to transfer the product from a stationary vessel to a bag, which can then be transported to some other location. Before being used, the vessel, the bag, and the connection therebetween must be sterilized. The bag and connection tubing are typically sterilized at the place of manufacture and are placed within individual packaging which protects their sterility during shipment. However, when the packaging is opened at the location where the bag is to be filled, unless this is done in a sterile environment, the bag and the connection tubing lose their sterility. Usually, clamps are applied to the bag and the tubing so that the interiors thereof remain sterile, but the interiors distally of the clamps lose their sterility when the packaging is opened. These interiors then have to be re-sterilized before the bag can be filled. It would therefore be desirable to have a method for providing a sterile connection between a vessel and a bag, as well as an assembly which can be used with such a method.

### SUMMARY OF THE INVENTION

According to the present invention, there is provided a sterilized assembly adapted to provide a sterile connection between a vessel and a bag. The assembly comprises the bag and collapsible tubing having three arms connected at a central manifold. A first of the arms is secured to the bag with the interior of the tubing in communication with the interior of the bag. The assembly further comprises a first selectively tightenable clamp member on the first tubing arm and a second selectively tightenable clamp member on a second of the tubing arms.

According to the present invention, a method for providing a sterile connection between a first vessel and a second vessel, wherein each of the first and second vessels has a respective connection port, comprises the steps of providing a collapsible connecting tube having first and second ends, providing a first selectively tightenable clamping member on the connecting tube between the first and second ends, securing the first end of the connecting tube to the connection port of the first vessel, tightening the first clamping member to collapse the connecting tube, sterilizing the first vessel and the connecting tube, securing the second end of the connecting tube to the connection port of the second vessel, sterilizing the interior of the second vessel and the interior of the connecting tube from its second end to where it is collapsed by the first clamping member, and loosening the first clamping member to dilate the connecting tube.

In accordance with an aspect of this invention, the step of sterilizing the interior of the second vessel and the interior of the connecting tube comprises the step of utilizing steam to perform the sterilization.

### BRIEF DESCRIPTION OF THE DRAWINGS

The foregoing will be more readily apparent upon reading the following description in conjunction with the drawings in which like elements in different figures thereof are identified by the same reference numeral and wherein:

FIG. 1 is a plan view of an embodiment of the assembly according to the present invention;

FIG. 2 is a view of the assembly shown in FIG. 1 connected to the vessel and to a steam trap;

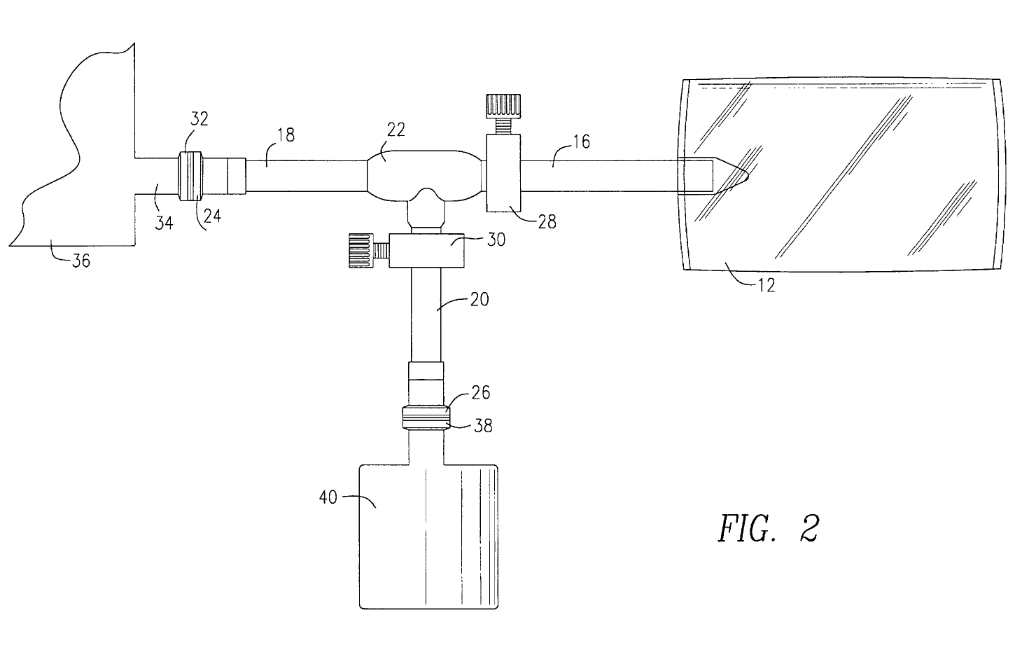

FIG. 3 is a flow chart of a sterilization method according to the present invention.

### DETAILED DESCRIPTION

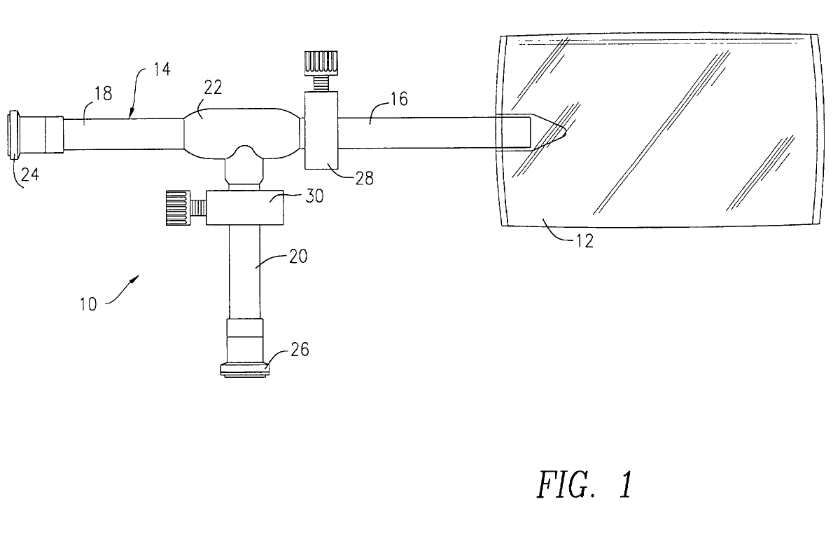

Referring now to the drawings, FIG. 1 shown an assembly, designated generally by the reference numeral 10, which may be utilized in practicing the present invention. The assembly 10 includes a bag 12 connected to collapsible connection tubing 14. The tubing 14 has three arms 16, 18, and 20 connected at a T-shaped central manifold 22. Fittings 24, 26 are secured to the distal ends of the tubing arms 18, 20, respectively, to allow for connection to corresponding fittings, as is conventional in the art. A first selectively tightenable clamp member 28 is provided on the tubing arm 16 and a second selectively tightenable clamp member 30 is provided on the tubing arm 20. It is preferred that the clamps 28, 30 be as close to the manifold 22 as possible. Specifically for the clamp member 28 this limits the intrusion of steam, as will be described hereinafter. Preferably, the bag 12 and the tubing 14 are both made of silicone and the interior of the bag 12 is in communication with the interior of the tubing 14. With the clamp member 28 tightened to collapse the tubing arm 16 and provide a seal thereat, the assembly 10 is sterilized, illustratively by radiation, and placed in a sealed package (not shown), so that it can be transported while remaining sterile.

When the assembly 10 arrives at the location where the bag 12 is to be filled, the first clamp member 28 remains closed and the assembly 10 is removed from its sterile package. The fitting 24 at the end of the tubing arm 18 is secured to a corresponding fitting 32 at the end of a connection port 34 of the vessel 36. A clamp, not shown, is used to secure the fitting 24 to the fitting 32. The fitting 26 is secured by a clamp, not shown, to a fitting 38 of a steam trap 40. After making sure that the second clamp member 30 is opened, steam is utilized to sterilize the interior of the vessel 36. This steam passes through the connection port 34 to the interior of the tubing arm 18 and the interior of the tubing arm 16 up to the first clamp member 28, thereby sterilizing this interior. The steam passes through the tubing arm 20, sterilizing its interior as well, and is collected in the steam trap 40. After this sterilization process, the second clamp member 30 is closed to seal off the tubing arm 20. Material may then be placed in the vessel 36 and the first clamp member 28 may be opened to allow the material to fill the bag 12. After the bag 12 is appropriately filled, the first clamp member 28 is closed to seal the material within the bag 12. The clamps on the fittings 24, 26 may then be removed so that the steam trap 40 can be removed and the now-filled bag 12, along with the tubing 14, is separated from the vessel 36.

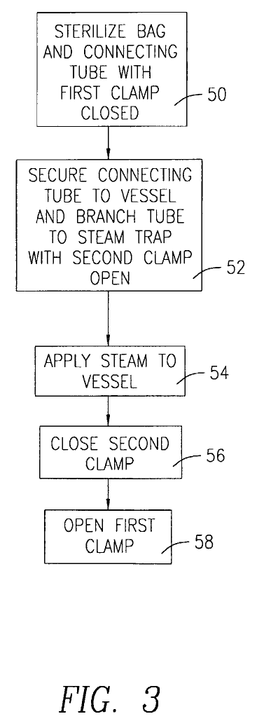

The aforedescribed sterilization method is illustrated in FIG. 3. As shown in the box 50, the bag 12 and the tubing 14 are sterilized with the first clamp 28 closed. Then, as shown in the box 52, the tubing 14 is connected to the vessel 36 and the tubing arm 20 is connected to the steam trap 40, with the second clamp 30 being open. As shown in the box 54, steam is applied to the vessel 36. As shown in the box 56, the second clamp 30 is closed. As shown in the box 58, the first clamp 28 is opened.

While the aforedescribed sterilization method has disclosed the use of steam for sterilizing the vessel 36 and the tubing 14, there may be other ways of sterilizing the vessel 36 and the tubing 14. In this case, the steam trap 40 would be unnecessary and the tubing 14 can be constructed as a straight tube without the manifold 22 or the tubing arm 20. The clamp member 28 would still be required and the assembly of the bag and tubing would be sterilized and then shipped in its sealed packaging with the clamp member 28 closed. In this case, the clamp member 28 would be as far from the bag 12 as possible.

Accordingly, there has been disclosed an improved method for providing a sterile connection between two vessels, along with an assembly that can be utilized in practicing the method. While an illustrative embodiment of the present invention has been disclosed herein, it is understood that various modifications and adaptions to the disclosed embodiment are possible, and it is intended that this invention be limited only by the scope of the appended claims.

What is claimed:

1. A method for providing a sterile connection between a first vessel and a second vessel, each of said first and second vessels having a respective connection port, comprising the steps of:
providing a collapsible connecting tube having first and second ends;
providing a first selectively tightenable clamping member on said connecting tube between said first and second ends;
securing the first end of said connecting tube to the connection port of said first vessel;
tightening said first clamping member to collapse said connecting tube;
sterilizing said first vessel and said connecting tube;
securing the second end of said connecting tube to the connection port of said second vessel;
sterilizing the interior of said second vessel and the interior of said connecting tube from its second end to where it is collapsed by said first clamping member; and
loosening said first clamping member to dilate said connecting tube.

2. The method according to claim 1 wherein the step of sterilizing the interior of said second vessel and the interior of said connecting tube comprises the step of:
utilizing steam to perform the sterilization.

3. The method according to claim 2 wherein:
the step of providing a connecting tube includes the step of providing a collapsible main connecting tube having first and second ends and a branch tube between said first and second ends, wherein said branch tube has a first end providing communication between the interior of said main connecting tube and the interior of said branch tube and a distal end; and
the step of providing a first clamping member includes the step of providing a first selectively tightenable clamping member on said connecting tube between said branch tube and said first end of said main connecting tube.

4. The method according to claim 3 further comprising the steps of:
providing a steam trap; and
securing the steam trap to the distal end of said branch tube to be in communication with the interior of said branch tube.

5. The method according to claim 4 further comprising the steps of:
providing a second selectively tightenable clamping member on said branch tube;
loosening said second clamping member prior to the step of utilizing steam; and
tightening said second clamping member after the step of utilizing steam and prior to the step of loosening said first clamping member.

6. A sterilized assembly adapted to provide a sterile connection between a vessel and a bag, comprising:
said bag;
collapsible tubing having three arms connected at a central manifold, wherein a first of the arms is secured to said bag with the interior of said tubing in communication with the interior of said bag;
a first selectively tightenable clamp member on said first tubing arm; and
a second selectively tightenable clamp member on a second of said tubing arms.

7. The assembly according to claim 6 wherein said first clamp member is tightened to collapse said first tubing arm and provide a seal within said first tubing arm.
